(19) United States
(12) Patent Application Publication
(10) Pub. No.: US 2015/0359659 A1
(43) Pub. Date: Dec. 17, 2015

(54) FISTULA EXTENSION AND RELATED METHOD OF TREATING AN OPEN WOUND AROUND AN ACTIVE FISTULA

(71) Applicant: Richard Christian, Ashland, KY (US)

(72) Inventor: Richard Christian, Ashland, KY (US)

(21) Appl. No.: 14/739,394

(22) Filed: Jun. 15, 2015

Related U.S. Application Data
(60) Provisional application No. 62/013,068, filed on Jun. 17, 2014.

(51) Int. Cl. A61F 5/449; A61M 35/00; A61F 13/14

(52) U.S. Cl. CPC A61F 5/449; A61F 13/148; A61M 35/00

(57) ABSTRACT

An apparatus for treating an open wound around an active fistula. The apparatus includes: (1) a fistula extension; (2) an abdominal binder; and (3) an ostomy bag assembly removably attached to the fistula extension. The fistula extension is sealed to a patient's skin such that secretion from the open fistula is transferred to the ostomy bag assembly.

This application claims the benefit of U.S. Provisional Patent Application Ser. No. 62/013,068, filed on Jun. 17, 2014, the disclosure of which is incorporated herein by reference.

### TECHNICAL FIELD

This disclosure relates generally to a medical apparatus or device for treatment of a skin graft or other medical conditions and, more particularly, to a fistula extension and related method for more efficiently treating an open wound around an active fistula, such as an intestinal fistula by channeling secretion from the wound and/or fistula to an ostomy bag or pouch system for disposal.

### BACKGROUND

Ostomies are surgically created openings from the intestine to the skin with the most common being colostomies. Skin care and containment of ostomy output are major considerations in the overall rehabilitation of the patient who has undergone the surgery resulting in the creation of the ostomy. One particular form of treatment of an ostomy is a pouching system including a wafer and a pouch, which is designed to protect the skin, contain the effluent/stool and odor and remains securely attached to the patient's skin for a significant amount of time.

In contrast to an ostomy, a fistula is not intentionally or surgically created. Instead, a fistula is an abnormal connection between an organ, vessel, intestine or another structure. Fistulas typically originate from any part of the gastrointestinal (GI) tract. Generally, a fistula may be described as a tunnel connecting two cavities or hollow organs within the body. A fistula may develop for many reasons, including previous surgeries and complications from surgical procedures, such as skin grafts, diseases, such as Crohn's disease or they may result from injuries, infection, inflammation, trauma, open wounds, surgical wounds, abscesses and ulcers or bed sores.

Fistulas occur in all shapes and sizes and are often located on a person's lower abdomen. A common example of a fistula is a tract or channel connecting a patient's large bowel out to the surface of the skin of the lower abdomen. There are numerous types of fistula, including but not limited to intestinal fistula, perianal, enterovesciular and vaginal fistula. Unlike an ostomy where a medical professional is able to select the preferred site and surgically create the stoma to optimize the conditions for effective treatment, such as pouching, fistula sites are not selected by the medical professional and may occur in areas where pouching is often difficult. As a result, an open abdominal wound with an active fistula can be extremely difficult and challenging to manage even for trained medical professionals.

Treatment for fistulas typically varies depending on the cause and extent of the fistula, but often involves surgical intervention combined with antibiotic therapy. Although a fistula may be treated in different ways, the treatments are usually designed to protect the skin, contain the drainage, control the odor and provide comfort to the patient. Most known treatment methods are usually time-consuming resulting in lengthy hospital stays and require substantial work by the hospital staff, which prevents the patient from leaving the hospital and increases medical expenses to the patient. Two common types of treatment are the use of an aquarium dressing and a wound vacuum, which are adhesively attached to the patient. Not only do these treatment methods require hospitalization, which increases the cost to the patient, but they are not particular effective treatment methods as they are slow to heal and limit patient mobility.

Specifically, these known treatment methods require a plastic covering with tubes over the fistula as well as the application of skin protectants. Since the skin is not exposed to the environment, it is typically extremely slow to heal. In addition, the skin around the fistula opening must be continuous monitored and evaluated by professionals. The dressing around the fistula opening must be changed up to three times a day including sucking out the discharge, which is a time-consuming and difficult process even for trained professionals, such as the hospital staff. Under this type of treatment, the patient is essentially immobile and must be confined to the hospital bed for extensive periods of time. Due to the prolonged time for recovery, the patient is also susceptible to side effects, such as infection and sepsis.

Accordingly, a need is identified for an apparatus and method of treating an active fistula in an open abdominal wound more efficiently both in terms of cost and time as well as the patient's comfort and mobility. Specifically, the fistula apparatus and treatment method should allow a patient more mobility while being used. Furthermore, the apparatus and method should allow the skin around the fistula to be exposed to the environment to promote quicker healing of the fistula tract without fear of infection and without the need for daily dressing changes. As a result, the apparatus and method may lead to quicker recovery and less hospitalization (and expense) for patients having a fistula.

### SUMMARY

In accordance with one aspect of the disclosure, an apparatus for treating an open wound around an active fistula is provided. The apparatus includes: (1) a fistula extension; (2) an abdominal binder; and (3) an ostomy bag assembly removably attached to the fistula extension. The fistula extension is sealed to the open wound such that secretion from the active fistula is transferred to the ostomy bag assembly.

In one embodiment, the fistula extension may be a single piece construction and made of a rigid material, such as plastic or nylon. The rigid material is sterile. The fistula extension may be configured to be positionable around the active fistula such that the open wound around the active fistula is directly exposed to ambient air. The ostomy bag assembly may have a collection device and a wafer. The fistula extension may be made from injection molding or a lathe process.

In accordance with another aspect of the disclosure, a fistula extension for treating an open wound around an active fistula of a patient is provided. The fistula extension includes a base and a top portion molded to the base. The fistula extension is positionable on the patient such that the open wound around the active fistula is exposed to ambient air to promote faster healing.

In one embodiment, the base is substantially circular having a substantially flat bottom portion and a first opening. The top portion has an inner side wall and an outer side wall extending vertically from the base. The inner side wall and the outer side wall of the top portion form a second opening The first opening and the second opening may be aligned to create an unobstructed opening through an entirety of the fistula extension. The top portion has a flat top edge connecting the inner side wall and the outer side wall of the top portion.

In yet another aspect of this disclosure, a method for treating an open wound around an active fistula of a patient is disclosed. The method includes: (1) providing a fistula extension having an opening; (2) positioning the fistula extension around the open wound of the active fistula such that the open wound and active fistula is visible in the opening and exposed to ambient air; and (3) sealing the fistula extension to the patient, wherein the step of sealing includes injecting a medical paste on the fistula extension.

### BRIEF DESCRIPTION OF THE DRAWINGS

The accompanying drawings incorporated in and forming a part of the specification, illustrate several aspects of this disclosure, and together with the description serve to explain the principles of the disclosure. In the drawings:

FIG. 1 is an isometric top view of a fistula extension forming one aspect of the disclosure;

FIG. 2 is a front view of the fistula extension forming one aspect of the disclosure, the left side, right side and rear views are identical;

FIG. 3 is a top view of the fistula extension forming one aspect of the disclosure;

FIG. 4 is a bottom view of the fistula extension forming one aspect of the disclosure;

FIG. 5 is a front view of an ostomy bag and wafer/baseplate forming one aspect of the disclosure; and

FIG. 6 is a side view of an abdominal binder forming one aspect of the disclosure.

### DETAILED DESCRIPTION

In the following detailed description, reference is made to the accompanying drawings that form a part hereof, and in which is shown by way of illustration, specific embodiments in which the disclosure may be practiced. These embodiments are described in sufficient detail to enable those skilled in the art to practice the embodiments and like numerals represent like details in the various figures. Also, it is to be understood that other embodiments may be utilized and that process or other changes may be made without departing from the scope of the disclosure. The following detailed description is not to be taken in a limiting sense, and the scope of the disclosure is defined only by the appended claims and their equivalents. In accordance with the disclosure, a fistula extension is hereinafter described.

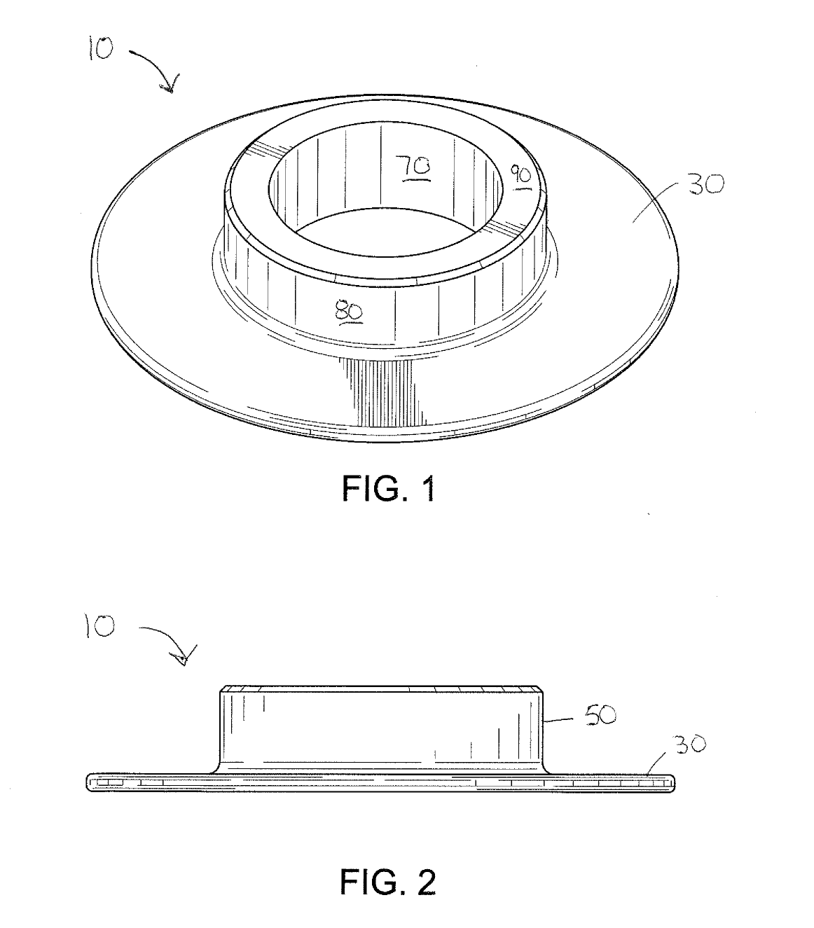

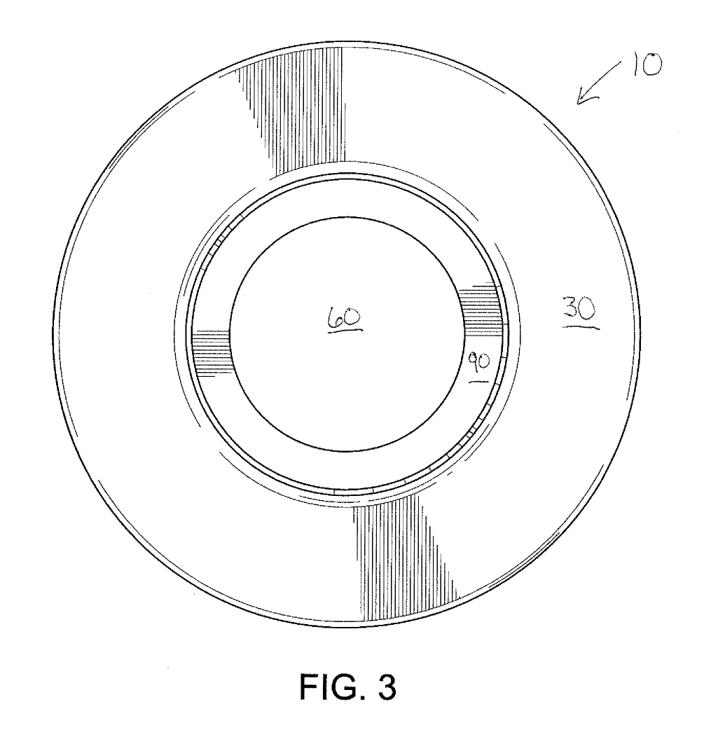

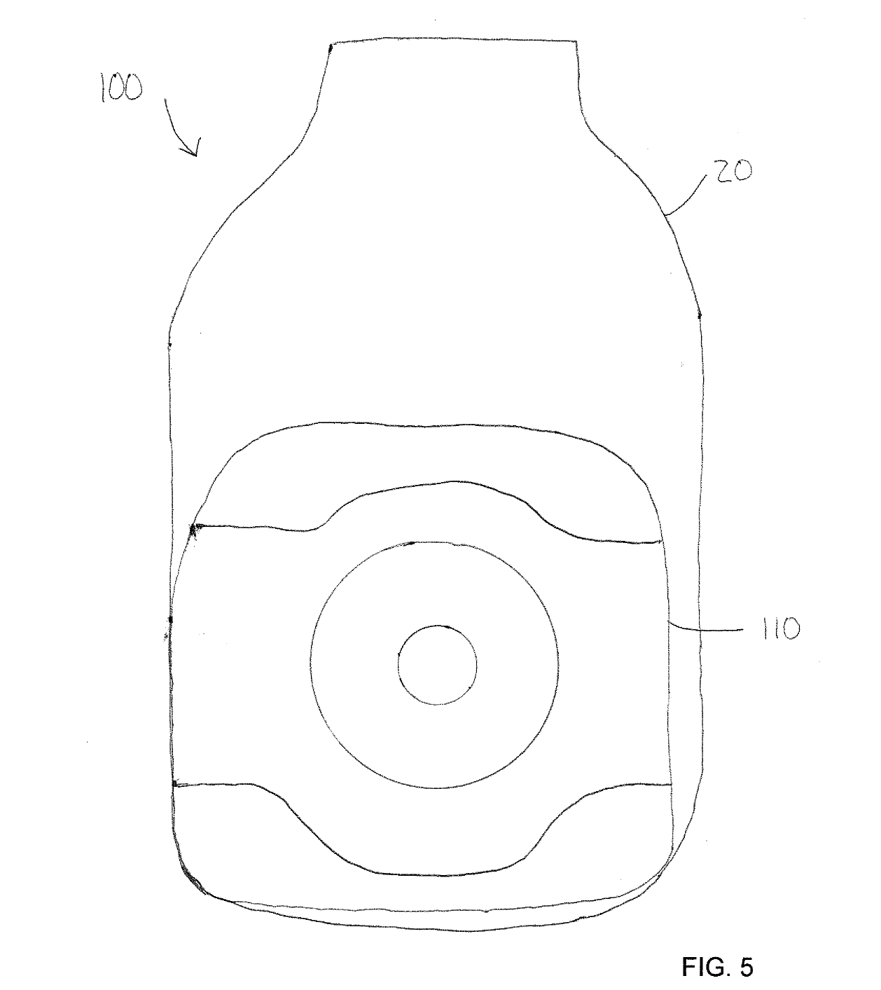

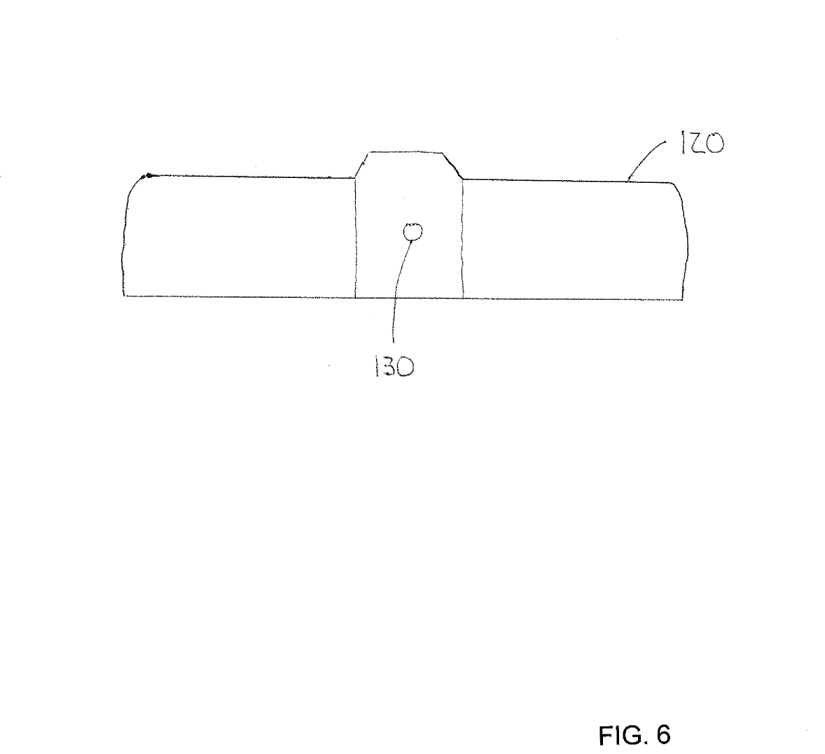

As shown in FIGS. 1-4, a fistula extension 10 for treatment of an active fistula in an open abdominal wound is illustrated. One of the primary purposes of the fistula extension 10 is to channel secretion from the open wound and/or intestinal fistula to an ostomy bag 20 (shown in FIG. 5) for disposal. Advantageously, the fistula extension 10 allows the wound or skin graft to remain open, i.e., exposed to the environment or ambient air for better circulation, which promotes faster healing. In other words, there is no covering directly over the wound at the fistula output as in prior art treatment methods of active fistula.

The fistula extension 10 is a single piece structure made of a rigid material for increased durability. The rigid material is typically made of a synthetic sterile material, such as hard plastic or any other plastic suitable for contact with the skin. Advantageously, the plastic material is sufficiently lightweight to allow the patient increased mobility. Since the fistula extension 10 is in direct contact with the patient's skin, the rigid material should be sterile and not have result in any adverse results via contact with the patient's skin. In one embodiment, the fistula extension 10 is made from food grain nylon. It should be appreciated that the fistula extension may be made of other plastic materials. The fistula extension 10 may be made by injection molding or from a lathe process.

In more detail, the fistula extension 10 has a substantially circular base 30. The base 30 has a substantially flat bottom portion so that it may be placed directly on the patient's skin substantially flush to the patient's body. In one particular embodiment, the base 30 has a width of approximately 0.125 inches and a diameter of approximately 6 inches. Of course, it should be appreciated that the fistula extension may be made in different sizes to accommodate the particular patient and patient's wound. The base 30 has an opening 40 in its center.

The fistula extension further has a top portion 50 protruding (or extending vertically) from the circular base 30. The top portion 50 also has an opening 60 in its center. The top portion 50 is centered approximately in the center of the base 30 such that the openings 40, 60 correspond to create a complete opening from the bottom of the fistula extension to the top of the fistula extension. In one embodiment, the total diameter of the second circular portion is approximately 3.5 inches and it has an inner diameter of approximately 2.375 inches. The height of the second circular portion is approximately 1 inch. Of course, it should be appreciated that the fistula extension made be made in different sizes to accommodate a particular patient and a particular type of medical condition.

The interior of the top portion 50 is hollow such that air may reach the open wound to promote quicker healing of the active fistula. The top portion 50 has an inner side wall 70 and an outer side wall 80. The inner side wall 70 forms the opening with the corresponding opening in the base portion while the outer side wall 80 contacts the top of the base portion 30. A flat top edge 90 of the top portion 50 extends between the inner side wall 70 and the outer side wall 80 of the top portion.

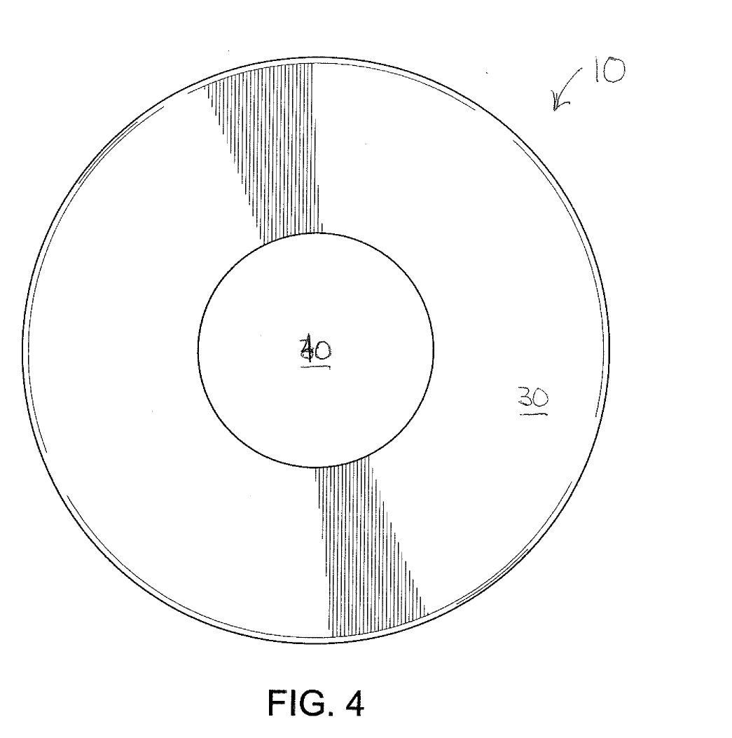

Turning to FIG. 4, the fistula extension 10 is connected to an ostomy bag assembly 100, which is a standard ostomy bag assembly known in the medical arts. Typically, the ostomy bag assembly 100 includes a collection device 20 such as an ostomy bag and a wafer, baseplate or ostomy flange 110. The wafer 110 is removably attached to the collection device or bag 20 such that the ostomy bag may be periodically emptied and cleaned as is common with the fluid output or drainage from the active fistula.

In use, the fistula extension 10 is prepared by attaching the wafer 110 to the top portion 50 of the fistula extension. The fistula extension 10 may be turned over and adapt paste or stoma paste is applied (typically from a tube) to the bottom of the fistula extension for sealing the fistula extension to the open wound. Specifically (as shown in FIG. 2), a bead of paste is placed on the top edge 90 of the fistula extension before it is placed on the open wound. Subsequently, the fistula extension 10 is placed on the open or active fistula and a syringe may be used to fill the interior of the fistula extension with paste. The paste is injected into the open area (between the two corresponding openings of the base and top portion of the fistula extension) around the fistula to protect the skin graft or other wound inside the fistula extension and to channel any secretion into the ostomy bag. A piece of silk tape (preferably 2 inch silk tape) may be applied over the top of the wafer and the bottom of the wafer to allow it to adhere to the outside of the wound. Thereafter, the ostomy bag 20 is attached to the wafer 110 by conventional means known in the art.

With reference to FIG. 5, an abdominal binder 120 is illustrated. The abdominal binder 120 may be a standard bandage or elasticized wrap applied around the lower part of the patient's torso to support the abdomen. The abdominal binder 120 is often applied after surgery to decrease discomfort and allow a patient to begin ambulatory activities to accelerate recovery. It may further improve blood circulation and oxygen levels at the operative site, increase healing and reduce swelling. As used herein, the abdominal binder 120 may be used in such a manner that a hole 130 is measured and cut into the soft padding to allow the ostomy bag to be pulled through the hole in the abdominal binder to the outside of the binder, i.e., over and around the whole abdomen of the patient. Advantageously, this allows the ostomy bag to be positioned on the outside of the binder so that it may emptied into a refuse container (not shown). The fistula extension 10 is secured to the patient such that is snug against the skin graft or wound to create a seal. However, the fistula extension 10 should not be so tight that the pressure on the skin graft or open wound ruptures or is excessively uncomfortable to the patient.

Advantageously, this apparatus and related method allows the skin graft or other type of open wound to heal quickly such that the intestines become pliable enough to do a reversal of the fistula in a shorter period of time than other procedures known in the art. Furthermore, this apparatus does not require as long a hospital stay as is it typically required for a treatment of an active fistula. Finally, this apparatus is relatively lightweight and easy to use, so the patient is not required to be in bed, but is able to be more mobile, which may also increase the healing time of the active fistula.

The foregoing descriptions of various embodiments are provided for purposes of illustration, and are not intended to be exhaustive or limiting. Modifications or variations are also possible in light of the above teachings. The embodiments described above were chosen to provide the best application to thereby enable one of ordinary skill in the art to utilize the disclosed inventions in various embodiments and with various modifications as are suited to the particular use contemplated. All such modifications and variations are within the scope of the disclosure.

1. An apparatus for treating an open wound around an active fistula, comprising:
a fistula extension;
an abdominal binder; and
an ostomy bag assembly removably attached to the fistula extension,
whereby the fistula extension is sealed to the open wound such that secretion from the active fistula is transferred to the ostomy bag assembly.

2. The apparatus of claim 1, wherein the fistula extension is a single piece construction.

3. The apparatus of claim 1, wherein the fistula extension is made of a rigid material.

4. The apparatus of claim 3, wherein the rigid material is plastic.

5. The apparatus of claim 3, wherein the rigid material is nylon.

6. The apparatus of claim 3, wherein the rigid material is sterile.

7. The apparatus of claim 1, wherein the fistula extension is configured to be positionable around the active fistula such that the open wound around the active fistula is directly exposed to ambient air.

8. The apparatus of claim 1, wherein the ostomy bag assembly including a collection device and a wafer.

9. The apparatus of claim 1, wherein the fistula extension is made from injection molding.

10. The apparatus of claim 1, wherein the fistula extension is made from a lathe process.

11. A fistula extension for treating an open wound around an active fistula of a patient, comprising:
a base; and
a top portion molded to the base,
whereby the fistula extension is positionable on the patient such that the open wound around the active fistula is exposed to ambient air to promote faster healing.

12. The fistula extension of claim 11, wherein the base is substantially circular.

13. The fistula extension of claim 11, wherein the base has a substantially flat bottom portion.

14. The fistula extension of claim 11, wherein the base has a first opening.

15. The fistula extension of claim 14, wherein the top portion has an inner side wall and an outer side wall extending vertically from the base.

16. The fistula extension of claim 15, wherein the inner side wall and the outer side wall of the top portion form a second opening.

17. The fistula extension of claim 16, wherein the first opening and the second opening are aligned to create an unobstructed opening through an entirety of the fistula extension.

18. The fistula extension of claim 15, wherein the top portion has a flat top edge connecting the inner side wall and the outer side wall of the top portion.

19. A method for treating an open wound around an active fistula of a patient, comprising:
providing a fistula extension having an opening;
positioning the fistula extension around the active fistula such that the open wound around the active fistula is visible in the opening and exposed to ambient air; and
sealing the fistula extension to the open wound of the patient.

20. The method of claim 19, wherein the sealing step includes injecting a medical paste on the fistula extension.
